(19) United States
(12) Patent Application Publication
(10) Pub. No.: US 2023/0381304 A1
(43) Pub. Date: Nov. 30, 2023

(54) NOVEL AGONIST VACCINE FORMULATION

(71) Applicant: BHARAT BIOTECH INTERNATIONAL LIMITED, Hyderabad (IN)

(72) Inventors: Krishna Mohan VADREVU, Hyderabad (IN); Ganneru BRUNDA, Hyderabad (IN); Krishna Murthy ELLA, Hyderabad (IN)

(73) Assignee: BHARAT BIOTECH INTERNATIONAL LIMITED, Hyderabad (IN)

(21) Appl. No.: 18/026,404

(22) PCT Filed: Sep. 15, 2021
(86) PCT No.: PCT/IN2021/050909

§ 371 Date: Mar. 15, 2023

(30) Foreign Application Priority Data
Sep. 15, 2020 (IN) 202041036825

(51) Int. Cl. A61K 39/39; A61K 39/215; A61K 47/10; A61P 31/14

(52) U.S. Cl. CPC A61K 39/39; A61K 39/215; A61K 47/10; A61P 31/14; A61K 2039/55511

(57) ABSTRACT

The invention relates to novel agonist vaccine formulation, wherein the agonist is novel TLR7/8 agonist which is used as an adjuvant or an immunomodulator. More particularly, the invention relates to the preparation of vaccine formulations against viral infections using Algel-IMDG as an adjuvant. The invention also relates to development of vaccine formulations for severe viral infections using the novel Algel-IMDG as an adjuvant that comprises TLR 7/8 agonist chemisorbed on to surface of Aluminium hydroxide gel. The invention also relates to the use of novel Algel-IMDG formulation as an adjuvant in Vaccine composition against several other viral diseases like Covid-19 caused by SARS-CoV-2 either wild type or its variants, Japanese Encephalitis, recombinant Hepatitis B surface antigen etc.

### RELATED PATENT APPLICATION

This application claims the priority to and benefit of Indian Patent Application No. 202041036825 filed on Sep. 15, 2020; the disclosures of which are incorporated herein by reference.

### FIELD OF THE INVENTION

The invention relates to the field of immunology, particularly adjuvants or vaccine formulation. More particularly, the invention relates to the preparation of vaccine formulations against viral infections using Algel-IMDG as an adjuvant. The invention also relates to the field of preparation of Algel-IMDG that comprises TLR 7/8 agonist chemisorbed on to surface of Aluminium hydroxide gel. The invention also relates to the use of novel Algel-IMDG formulation as an adjuvant in Vaccine composition against several other viral diseases like Covid-19 caused by SARS CoV 2 either wild type or its variants, Japanese Encephalitis, recombinant Hepatitis B surface antigen etc

### BACKGROUND OF THE INVENTION

Over the few decades, various approaches such as inactivated vaccines, viral vectored platform, live attenuated, subunit vaccine have been used to develop preventive vaccines against the infectious diseases (https://www.hhs.gov/immunization/basics/types/index.html). Most of the successful vaccine approaches aimed to produce more of humoral responses rather than T cell responses. Furthermore, refinement and simplification, and the use of subunit vaccines to increase the ease of manufacturing capabilities and also safety of the vaccine, lead to decrease in the vaccine potency. Hence, vaccine approaches need further development, in the need of T cell responses, especially to combat complex diseases such as tuberculosis, Malaria, SARS, MERS, Zika, Dengue and the most recent novel coronavirus disease, namely COVID-19. In an attempt to improve vaccine approaches for these complex diseases, several immunomodulators came into light as an adjuvant molecule [Gilbert, S. C. (2011), T-cell-inducing vaccines—what's the future. Immunology, 135, 19-26]. Immunomodulators or adjuvants are substances that enhance antigen specific immune responses, when used along with antigen. Adjuvants also serve to reduce the amount of antigen needed for the induction of a robust immune response (‘dose-sparing effect’) or the number of immunizations needed for protective immunity. Adjuvants also help to improve the efficacy of vaccines in new-borns, the elderly or immunocompromised persons, or can be used as antigen delivery systems for the uptake of antigens ([Reed, S. G.; Orr, M. T.; Fox, C. B., Key roles of adjuvants in modern vaccines. Nature medicine 2013, 19 (12), 1597-608] & [Coffman, R. L.; Sher, A.; Seder, R. A., Vaccine adjuvants: putting innate immunity to work. Immunity 2010, 33 (4), 492-503]). Further, the ability of adjuvants to broaden antibody responses could be more crucial for the efficacious vaccines against pathogens that display substantial antigenic drift and/or strain variations including influenza viruses, human immunodeficiency virus (HIV), human papilloma virus (HPV), and the malaria parasite ([Wiley, S. R.; Raman, V. S.; Desbien, A.; Bailor, H. R.; Bhardwaj, R.; Shakri, A. R.; Reed, S. G.; Chitnis, C. E.; Carter, D., Targeting TLRs expands the antibody repertoire in response to a malaria vaccine. Science translational medicine 2011, 3 (93), 93ra69]; [O'Hagan, D. T.; Valiante, N. M., Recent advances in the discovery and delivery of vaccine adjuvants. Nature reviews. Drug discovery 2003, 2 (9), 727-35]; [Mosca, F.; Tritto, E.; Muzzi, A.; Monaci, E.; Bagnoli, F.; Iavarone, C.; O'Hagan, D.; Rappuoli, R.; De Gregorio, E., Molecular and cellular signatures of human vaccine adjuvants. Proceedings of the National Academy of Sciences of the United States of America 2008, 105 (30), 10501-6]; [McKee, A. S.; Munks, M. W.; Marrack, P., How do adjuvants work? Important considerations for new generation adjuvants. Immunity 2007, 27 (5), 687-90.)]).

Innate immune system is the first line of defence mechanism, and it is essential to fight against severe diseases like SARS-CoV-2, MERS, SARS, etc. Innate immune system gets activated by binding of immunomodulators (known as Pathogen Associated membrane receptors), to the pattern recognition receptors (PRRs). Upon PRR activation, it triggers cascade of events to induce adaptive immunity to protect against pathogens or infections, by secreting cytokines, mainly type I/III interferons and also pro-inflammatory cytokines [Akira S., Takeda K. Toll-like receptor signalling. Nat. Rev. Immunol. 2004; 4: 499-511]. Several of these immunomodulators are being used as adjuvants in vaccines to stimulate Th1 response and also to get dose sparing effect of vaccine ([Akira, S.; Uematsu, S.; Takeuchi, O., Pathogen recognition and innate immunity. Cell 2006, 124 (4), 783-801]; [Kumagai, Y.; Takeuchi, O.; Akira, S., Pathogen recognition by innate receptors. Journal of infection and chemotherapy: official journal of the Japan Society of Chemotherapy 2008, 14 (2), 86-92]; [Kawai, T.; Akira, S., The role of pattern-recognition receptors in innate immunity: update on Toll-like receptors. Nat Immunol 2010, 11 (5), 373-84]). Imidazoquinoline, belongs to a class of PRRs which binds to endosomal transmembrane toll-like receptors 7 and 8 (TLR7 and TLR8) receptor and stimulate T cells, after the release of pro-inflammatory cytokines [M J Reiter et al., 1994. Cytokine induction in mice by the immunomodulator imiquimod. J Leukoc Biol 1994 February; 55(2):234-40. doi: 10.1002/jlb.55.2.234].

Several of TLR7 and TLR8 agonists and antagonists ligands have been used for therapeutic purposes [Patinote, C., et al., 2020. Agonist and antagonist ligands of toll-like receptors 7 and 8: Ingenious tools for therapeutic purposes. Eur J Med Chem. 2020 May 1; 193: 112238]. Till now, there is no commercial vaccine available for human use with TLR7/8 agonist. However, similar imidazoquinoline class molecules have been tested in both animal models and in human clinical trials for several purposes either as an adjuvant or as an immunotherapeutic molecule. Especially, during COVID-19 pandemic, several TLR agonists have been used to treat COVID-19 patients [Florindo, H F et al., 2020. Immune-mediated approaches against COVID-19. Nature Nanotechnology, Vol 15, August 2020, 630-645]; [Angelopoulou et al., Imiquimod—A toll like receptor 7 agonist—Is an ideal option for management of COVID 19. Environmental ResearchVolume 188, September 2020, 109858 https://doi.org/10.1016/j.envres.2020.109858]; [Poulas, K et al., 2020. Activation of TLR7 and Innate Immunity as an Efficient Method Against COVID-19 Pandemic: Imiquimod as a Potential Therapy. Front Immunol. 2020; 11: 1373. doi: 10.3389/fimmu.2020.01373]).

It is known that vaccine should be safe, effective and furthermore it should also minimize the antibody dependent enhancement (ADE) especially in the case of most severe diseases caused by the Coronaviruses and Flaviviruses such as COVID-19, SARS, MERS, Zika virus and Japanese Encephalitis viruses. Though, there has been several approaches such as whole inactivated, subunits, viral vectored platforms, DNA, RNA and virus-like particles (VLPs) have all been tested are being developed and are at various stages of developmental stage.

In order to increase the efficacy of the vaccine, while minimizing the Antibody dependent enhancement (ADE) effect, present invention discloses use of Algel-IMDG® as an adjuvant along with inactivated whole virion SARS-CoV-2 vaccine (BBV152 A & B) and compared with Algel (BBV152C). The present invention discloses use of m-Amine Gallamide N-(3-((4-amino-2-butyl-1H-imidazo[4,5-c]quinolin-1-yl) methyl) benzyl)-3,4,5-trihydroxybenzamide, a novel TLR7/8 agonist, chemisorbed on to the surface of aluminium hydroxide and this preparation is named as Algel-IMDG. This technology was Immidazoquinoline licensed from Virovax, USA. The present invention focusses on the use of this adjuvant (Algel-IMDG) to test with different vaccines to increase the efficacy of the vaccines, both in terms of humoral and cell mediated immunity, while minimizing the Antibody dependent enhancement (ADE).

### OBJECTS OF THE INVENTION

The primary object of the invention is to provide novel agonist vaccine formulation, wherein the agonist is novel TLR7/8 agonist which is used as an adjuvant or an immunomodulator.

Another object of the invention is to prepare adjuvant formulation (Algel-IMDG) using novel TLR7/8 agonist for viral vaccines.

Another object of the invention is to provide preparation of novel Algel-IMDG that comprises TLR 7/8 agonist chemisorbed on to surface of Aluminium hydroxide gel.

Another object of the invention is to provide vaccine formulations using chemisorbed Aluminium hydroxide with TLR7/8 agonist molecule as adjuvant.

Another object of the invention is development of vaccine formulations for severe viral infections using this adjuvant formulation i.e. chemisorbed Aluminium hydroxide with TLR7/8 agonist as an adjuvant to increase the effectiveness of the vaccine.

Yet another object of the invention is to test said adjuvant formulation (Algel-IMDG) with different vaccines to increase the efficacy of the vaccines, both in terms of humoral and cell mediated immunity while minimizing the Antibody dependent enhancement (ADE).

It is an object of the invention to evaluate the long-term immunity of the vaccine formulation comprising novel Algel-IMDG, in terms of Spike specific antibody titers and neutralization antibody titers.

A further object of the invention is to develop highly safe and effective vaccine formulations against severe viral infections using this novel agonist.

### SUMMARY OF THE INVENTION

The present invention relates to novel agonist vaccine formulation, wherein the agonist is novel TLR7/8 agonist which is used as an adjuvant or an immunomodulator.

Accordingly, in one aspect the present invention provides novel adjuvant for vaccines using this novel agonist.

In another aspect, the invention provides development of vaccine formulations for severe viral infections using novel adjuvants based on this agonist.

A vaccine formulation for prophylactic vaccine against viral infections, comprising:
(a) a vaccine antigen;
(b) Algel-IMDG as an adjuvant;
(c) preservative; and
(d) a physiologically acceptable buffer.

In the said vaccine formulation, the vaccine antigen is a whole virion inactivated SARS-CoV-2 or SARS-CoV-2 variants selected form B.1.617.2 (Delta), Brazilian variant (P.1), South African S.501Y.V2 (also known as B.1.351), Japanese Encephalitis (JE), recombinant Hepatitis B surface antigen or Virus like particles (VLPs) such as Human papilloma virus antigen.

The said vaccine antigen SARS-CoV-2, SARS-CoV-2 variants or JE is inactivated by beta propiolactone or formaldehyde.

The concentration of said vaccine antigen SARS-CoV-2, SARS-CoV-2 variants or JE in the said formulation is 1 to 20 μg.

In the said vaccine formulation, Algel-IMDG comprises Al gel as delivery system and Toll-like receptor 7 and Toll-like receptor 8 agonist as a small molecule (IMDG) that can activate immune cells.

The Al gel is Aluminium hydroxide gel or Aluminium phosphate gel.

The Toll-like receptor 7 and Toll-like receptor 8 agonist is meta-amine gallamide N-(3-((4-amino-2-butyl-1H-imidazo[4,5-c]quinolin-1-yl) methyl) benzyl)-3,4,5-trihydroxybenzamide.

In the said vaccine formulation, Algel-IMDG comprises meta-amine gallamide N-(3-((4-amino-2-butyl1H-imidazo[4,5-c]quinolin-1-yl) methyl) benzyl)-3,4,5-trihydroxybenzamide (Imidazoquinoline class molecule), chemisorbed with Aluminium hydroxide gel.

The said Toll-like receptor 7 and Toll-like receptor 8 agonist with functional groups allow the chemisorption of such compounds to the surface of aluminium hydroxide particles.

In the said vaccine formulation, the Algel-IMDG is prepared by allowing the chemisorption of meta-amine gallamide on to the surface of aluminium hydroxide particles, under continuous stirring upto 72 hrs, allowing the targeted delivery of the Toll-like receptor 7 and Toll-like receptor 8 agonist to draining lymph nodes with negligible systemic exposure, resulting in minimal systemic reactogenicity.

The Algel-IMDG is prepared by the method comprising the steps of.
(i) dissolving meta-amine gallamide N-(3-((4-amino-2-butyl-1H-imidazo[4,5-c]quinolin-1-yl) methyl) benzyl)-3,4,5-trihydroxybenzamide in isopropanol;
(ii) keeping the solution of step (i) at 50° C. to dissolve completely;
(iii) filtering the solution of step (ii); and
(iv) adding the solution of N-(3-((4-amino-2-butyl-1H-imidazo[4,5-c]quinolin-1-yl) methyl) benzyl)-3,4,5-tri-hydroxybenzamide obtained from step (iii) to Aluminium hydroxide gel, dropwise under continuous stirring for 72 hours to obtain chemisorbed meta-amine gallamide on to the surface of aluminium hydroxide particles (Algel-IMDG).

In the said vaccine formulation, Algel-IMDG comprises 600-1000 μg of TLR7/8 agonist per ml of Algel-IMDG.

In the said vaccine formulation, Algel-IMDG comprises 250-750 μg of Al³⁺ concentration per dose in 0.5 ml.

Further in the said vaccine formulation, Algel-IMDG comprises 15-25 μg of TLR7/8 agonist per dose in 0.5 ml.

The preservative in the said vaccine formulation is Thimerosal or 2-phenoxy ethanol.

The concentration of Thimerosal in the formulation is 0.003 to 0.01%.

The concentration of 2-phenoxy ethanol in the formulation is 1 to 5 mg/ml.

The buffer used in the said vaccine formulation is phosphate or citrate.

The said formulation is stable for 12 months at 2-8° C., 6 months at 25±2° C. and upto 14 days at 37±0.2° C.

The formulation provides long-term protective immunity upto 7 months (6 months, post 2^{nd} dose) to the virus by generation of B and T cell memory responses in the vaccinated individuals.

The said formulation provides cross neutralization against SARS-CoV-2 variants such as homologous strain (D614G) and heterologous strains such as B.1.128.2, B.1.351, B.1.1.7, B.617, B.617.2.

The formulation of the present invention is used for prophylactic or therapeutic purposes.

In a further aspect, the invention uses the novel agonist-based adjuvants for vaccine formulations against severe viral disease like Covid-19 caused by SARS-CoV-2 either wild type or its variants, Japanese Encephalitis, recombinant Hepatitis B surface antigen etc.

Yet in another aspect the invention uses this novel adjuvant to test with different vaccines to increase the efficacy of the vaccines, both in terms of humoral and cell mediated immunity while minimizing the Antibody dependent enhancement (ADE).

### BRIEF DESCRIPTION OF DRAWINGS

FIG. 1: Structure of IMDG (Example 1.1): N-(3-((4-amino-2-butyl-1H-imidazo[4,5-c]quinolin-1-yl) methyl) benzyl)-3,4,5-tri-hydroxybenzamide.

FIG. 2(A): Physico-chemical properties of IMDG (Meta amine Gallamide) (Example 1.1) shown via NMR—Nuclear Magnetic Resonance.

FIG. 2(B): LC-MS/MS of IMDG (Meta-amine Gallamide).

FIG. 2(C): Mol. Wt of IMDG by LC-MS.

FIG. 2(D): Purity of IMDG by HPLC—High performance Liquid Chromatography.

FIG. 3: Depicts the Diagramatic representation of chemisorbed IMDG onto the Aluminium hydroxide gel, namely Algel-IMDG (Example 1.2).

FIG. 4: Illustrates the bioactivation of Algel-IMDG as shown by the release of IFN-alpha from the cell culture supernatant, when stimulated PBMCs with Algel-IMDG or Adjuvanted vaccine formulations (BBV152 A, B & C) for 48 hrs. Absorbance obtained from the unstimulated cell culture supernatant was taken as background (Example 3).

FIG. 5A: Represents dose sparing effect of Algel-IMDG® in Inactivated Whole Virion SARS-CoV-2 antigen (Example 4.2).

FIG. 5B: Represents dose Sparing effect of Algel-IMDG® compared with antigen alone (Example 4.2).

FIG. 6: Immunoglobulin Subclass (Example 5.1).

FIG. 7: Sars-CoV-2 Cell-Mediated Responses (Example 5.2).

FIG. 8: Humoral response in Syrian Hamsters (Example 6):

(A) IgG antibody response for all groups of animals observed on 12, 21, and 48 days.

(B) IgG antibody response at post-infection (3, 7, and 15 DPI) for all groups of animals.

(C) Comparison of IgG antibody titers between groups on 12, 21, and 48 days.

(D) Comparison of IgG antibody titers between groups after virus challenge at 3, 7, and 15 DPI.

(E) Comparison of IgG2 antibody titers between groups during immunization period at 21 and 48 days and post virus challenge at 3, 7, and 15 DPI (F and G).

(F) Comparison of NAb titers response during a three-dose vaccine regime for all groups of animals observed on 12, 21, and 48 days.

(G) Comparison of NAb titers response in SARS-CoV-2 infected animals on 3, 7, and 15 DPI. Mean along with standard deviation (SD) is depicted in the scatterplot. The statistical significance was assessed using the Kruskal-Wallis test followed by the two-tailed Mann-Whitney test between the two groups; p values less than 0.05 were considered to be statistically significant. The dotted lines indicate the limit of detection of the assay

FIG. 9: Gross and histopathological observations of lungs in hamsters after virus inoculation (Example 6):

(A-D) (A) Lungs of hamster from group I on 7 DPI showing diffuse areas of consolidation and congestion in the left and right lower lobe with few congestive foci in right upper lobe, scale bar=0.73 cm. Lungs from (B) group II, scale bar=0.65 cm (C) group III, scale bar=0.73 cm and (D) group IV showing normal gross appearance on 7 DPI scale bar=0.52 cm.

(E) Lung tissue from group I on 3 DPI showing acute inflammatory response with diffuse alveolar damage, haemorrhages, inflammatory cell infiltration (black arrow), hyaline membrane formation (white arrow), and accumulation of eosinophilic edematous exudate (star), scale bar=20 mm.

(F) Lung tissue of group I on 7 DPI showing acute interstitial pneumonia with marked alveolar damage, thickening of alveolar and accumulation of mononuclear cells, and macrophages (white arrow), and lysed erythrocytes in the alveolar luminal space (star), scale bar=20 mm.

(G-J) (G) Lung tissue from group I on 15 DPI depicting interstitial pneumonia with marked thickening of alveolar septa with type-II pneumocyte hyperplasia and fibro-elastic proliferation with collagen deposition at alveolar epithelial lining (white arrow), scale bar=20 mm. Lung section from group II showing no evidence of disease (H) on 3 DPI few congestive foci, scale bar=20 mm (I) on 7 DPI, scale bar=20 mm (J) on 15 DPI, scale bar=20 mm.

FIG. 10: Gross pathology of lungs of vaccinated and unvaccinated Non-human primates, after live virus challenge (Example 6):

(a) Lungs showing extensive involvement of the right upper lobe (RUL), right lower lobe (RLL), left upper lobe (LUL) and left lower lobe (LLL) (group I), and

(b) normal lung (group III).

FIG. 11: Long term immune response elicited against adjuvanted vaccine formulations in BALB/c mice (Example 7):

(A) Spike specific antibody titers by ELISA, and

(B) Neutralization antibody titers by MNT₅₀, shown upto 98 days, post vaccination.

FIG. 12: T cell memory response (Example 10).

FIG. 13: Th1 biased cytokine response indicative of activation of adaptive immune response (Example 9).

FIG. 14: Memory B cell response with secreting IgG & IgA response (Example 11).

FIG. 15: Cross Neutralization antibodies shown by the Inactivated SARS-CoV-2 antigen formulated with Algel-IMDG (Example 12).

FIG. 16: Efficacy of BBV152B (Inactivated SARS-CoV-2 antigen formulated with Algel-IMDG) against SARS-CoV-2 variants (Example 13).

### DETAILED DESCRIPTION OF THE INVENTION

Present invention discloses novel agonist vaccine formulation, wherein the agonist is novel TLR7/8 agonist which is used as an adjuvant or an immunomodulator.

The present invention discloses the use of novel chemisorbed TLR7/8 agonist molecule in preparation of adjuvants for vaccine formulations. The invention discloses the use of novel chemisorbed TLR7/8 agonist molecule into Aluminium hydroxide (Algel-IMDG) as an adjuvant to increase the effectiveness of the vaccine.

Throughout the description, tables and drawings, wherever used, the expression Algl-IMDG/Algel-IMDG represents the novel adjuvant of the invention. Expressions “novel adjuvant” or “Algl-IMDG”/“Algel-IMDG” invariably used throughout the description and drawings will have the same meaning and represent the same novel product of the invention. Wherever used the expressions singular like adjuvant, formulation, vaccine or plural like adjuvants, formulations, vaccines have the same meaning.

Accordingly, in one aspect, the invention provides novel agonist for vaccines. In another aspect, the invention provides novel adjuvant for vaccines using this novel agonist. In a further aspect, the invention provides development of vaccine formulations for severe viral infections using novel adjuvants based on this agonist. In a further aspect, the invention uses the novel agonist-based adjuvants for vaccine formulations against severe viral disease like Covid-19, Japanese Encephalitis, Zika, MERS, SARS etc.

### Novel Adjuvant of the Invention: Algel-IMDG

A potential drawback of using TLR agonists, small molecule as vaccine adjuvants is their ability to diffuse out of the injection site into systemic circulation. This tendency not only limits their adjuvant property but also enhances the risk of systemic reactogenicity. This can lead to systemic side effects including fever, headache, malaise, and myalgia, likely due to systemic immune activation. Hence, to limit the systemic exposure, adsorbing small molecules onto the “alum” [Al(OH)₃] has been tried earlier, in order to minimize systemic exposure of the TLR agonist(s) while trafficking the delivery to draining lymph nodes.

In one aspect, the present invention is directed towards novel adjuvant comprising m-Amine gallamide N-(3-((4-amino-2-butyl1H-imidazo[4,5-c]quinolin-1-yl) methyl) benzyl)-3,4,5-trihydroxybenzamide, which is a novel Meta amine Gallamide (Imidazoquinoline class molecule), chemisorbed with Aluminium hydroxide.

The present invention comprises of m-Amine Gallamide N-(3-((4-amino-2-butyl-1H-imidazo[4,5-c]quinolin-1-yl) methyl) benzyl)-3,4,5-trihydroxybenzamide, a novel TLR7/8 agonist, chemisorbed on to the surface of aluminium hydroxide and this preparation is named as Algel-IMDG. This technology was Immidazoquinoline licensed from Virovax, USA.

In one embodiment the invention uses this adjuvant to test with different vaccines to increase the efficacy of the vaccines, both in terms of humoral and cell mediated immunity while minimizing the Antibody dependent enhancement (ADE).

The present invention discloses the use of novel agonist as an adjuvant along with the Whole inactivated SARS-CoV-2 and Japanese Encephalitis vaccine, wherein adjuvant is comprised of Aluminium hydroxide (Algel) chemisorbed with m-amine Gallamide, a novel synthetic TLR7/8 agonist.

In one embodiment, the present invention discloses adsorption characteristics of antigen to the novel agonist.

In another embodiment, it also discloses the bioactivity of the novel adjuvant either by in-vitro or Ex-vivo or in-vivo assays.

The novel Algel-IMDG of the present invention contains delivery system Aluminium hydroxide and Toll-like receptor 7 and Toll-like receptor 8 agonist, a small molecule (IMDG) that can activate immune cells. The Algel-IMDG is a novel Toll-like receptor 7 and Toll-like receptor 8 agonist with functional groups which allow the chemisorption of such compounds to the surface of aluminium hydroxide particles. Algel-IMDG may contain meta-amine gallamide or Imidazoquinoline class molecules or such derivatives, salt, tautomer, polymorph, solvate or combination thereof.

In the said adjuvant, the Algel can be either Aluminium hydroxide gel or Aluminium phosphate gel.

The said Algel-IMDG is a novel Toll-like receptor 7 and Toll-like receptor 8 agonist, also named as meta amine gallamide with a IUPAC name N-(3-((4-amino-2-butyl-1H-imidazo[4,5-c]quinolin-1-yl) methyl) benzyl)-3,4,5-trihydroxybenzamide.

The adjuvant of the present invention may contain 600-1000 μg of TLR7/8 agonist per ml of Algel-IMDG.

When used in the vaccine formulation, Algel-IMDG activates innate immunity, thereby helps the vaccine to induce both humoral and cell mediated responses to enhance the vaccine efficacy.

Algel-IMDG when used as an adjuvant in the vaccine formulation helps to enhance the immune response against antigen.

Algel-IMDG was found to be stable at 2-8° C. for upto 90 days, and accelerated temperatures such as at room temperature (25° C.) and 37° C., upto 15 days.

### Method for Preparation of Novel Adjuvant Algel-IMDG:

Another aspect of the present invention is to provide a method for preparation of Algel-IMDG.

A method for preparation of Algel-IMDG comprises the steps of:
(i) dissolving meta-amine gallamide N-(3-((4-amino-2-butyl-1H-imidazo[4,5-c]quinolin-1-yl) methyl) benzyl)-3,4,5-trihydroxybenzamide in isopropanol;
(ii) keeping the solution of step (i) at 50° C. to dissolve completely;
(iii) filtering the solution of step (ii); and
(iv) adding the solution of N-(3-((4-amino-2-butyl-1H-imidazo[4,5-c]quinolin-1-yl) methyl) benzyl)-3,4,5-tri-hydroxybenzamide obtained from step (iii) to Aluminium hydroxide gel, dropwise under continuous stirring for 72 hours to obtain chemisorbed meta-amine gallamide on to the surface of aluminium hydroxide particles (Algel-IMDG).

### Vaccine Formulation:

In another aspect, present invention is directed towards the vaccine formulation comprising chemisorbed Aluminium hydroxide with TLR7/8 agonist molecule as adjuvant.

The present invention discloses the Vaccine formulation with above-described novel adjuvant eliciting high antibody binding titers and also neutralizing antibody titers.

A vaccine formulation for prophylactic vaccine against viral infections, comprising:
(a) a vaccine antigen;
(b) Algel-IMDG as adjuvant;
(c) preservative; and
(d) a physiologically acceptable buffer.

(a) Vaccine antigen: The vaccine antigen in the said formulation comprises whole virion inactivated SARS-CoV-2 or SARS-CoV-2 variants such as B.1.617.2 (Delta), Brazilian variant (P.1), south African S.501Y.V2, also known as B.1.351 or Japanese Encephalitis (JE) or recombinant Hepatitis B surface antigen or Virus like particles (VLPs) such as Human papilloma virus antigen etc.

The said antigens of SARS-CoV-2 or SARS-CoV-2 variants or JE were inactivated by beta propiolactone or formaldehyde.

The concentration of antigens such as SARS-CoV-2 or SARS-CoV-2 variants or JE in the said formulation may range from 1 to 20 μg.

(b) Algel-IMDG as adjuvant: The said formulation comprises novel agonist as an adjuvant along with the Whole inactivated SARS-CoV-2 and Japanese Encephalitis vaccine, wherein adjuvant is comprised of Aluminium hydroxide (Algel) chemisorbed with m-amine Gallamide, a novel synthetic TLR7/8 agonist. Algel-IMDG helps to enhance the immune response against antigen.

The novel Algel-IMDG of the present invention contains delivery system Aluminium hydroxide and Toll-like receptor 7 and Toll-like receptor 8 agonist, a small molecule (IMDG) that can activate immune cells. The Algel-IMDG is a novel Toll-like receptor 7 and Toll-like receptor 8 agonist with functional groups which allow the chemisorption of such compounds to the surface of aluminium hydroxide particles. Algel-IMDG may contain meta-amine gallamide or Imidazoquinoline class molecules or such derivatives, salt, tautomer, polymorph, solvate or combination thereof. In the said adjuvant formulation, the Algel can be either Aluminium hydroxide or Aluminium phosphate gel.

The said Algel-IMDG is a novel Toll-like receptor 7 and Toll-like receptor 8 agonist, also named as meta amine gallamide with a IUPAC name N-(3-((4-amino-2-butyl-1H-imidazo[4,5-c]quinolin-1-yl) methyl) benzyl)-3,4,5-trihydroxybenzamide.

The Algel-IMDG was prepared by allowing the chemisorption of Meta amine Gallamide on to the surface of aluminium hydroxide particles, under continuous stirring upto 72 hrs. Such preparations allow the targeted delivery of the Toll-like receptor 7 and Toll-like receptor 8 agonist to draining lymph nodes with negligible systemic exposure, resulting in minimal systemic reactogenicity.

The adjuvant of the present invention may contain 600-1000 μg of TLR7/8 agonist per ml of Algel-IMDG.

In the said vaccine formulation, Algel-IMDG may contain 250-750 μg of Al³⁺ concentration per dose in 0.5 ml.

In the said vaccine formulation, Algel-IMDG may contain 15-25 μg of TLR7/8 agonist per dose in 0.5 ml.

When used in the vaccine formulation, Algel-IMDG activates innate immunity, thereby helps the vaccine to induce both humoral and cell mediated responses to enhance the vaccine efficacy.

(c) Preservatives: The vaccine formulation further comprises preservative such as Thimerosal or 2-phenoxy ethanol.

The concentration of 2-phenoxy ethanol in the said formulation can be ranged from 1 to 5 mg/ml.

The concentration of Thimerosal in the said formulation can be ranged from 0.003 to 0.01%.

(d) Buffer: The said formulation comprises a physiologically acceptable buffer selected from phosphate and citrate.

The vaccine formulation of the present invention helps to induce high Nab titers, along with SARS-CoV-2 specific (Spike, RBD & N protein) antibody binding titers by ELISA.

In the said formulation, Agel-IMDG showed better efficacy by providing early protection to animals (Syrian Hamster and NHP models) against SARS-CoV-2 infection. Agel-IMDG has been proved to be safe to use in humans with less solicited adverse events in the phase I clinical trials.

Further, in the said formulation the Agel-IMDG showed better T cell memory response with effector function indicated by the release of Th1 biased cytokines as determined in phase I & II clinical trials.

The said vaccine formulation used in phase II clinical trial showed B cell memory response with SARS-CoV-2 specific antibody secreting B cell suggestive of long-term immunity. The said vaccine composition used in phase II clinical trial shown that vaccine provides long term immunity tested upto 7 months (6 months, post 2^{nd} dose).

The said vaccine formulation used in phase III clinical trial shown cross neutralization against SARS-CoV-2 variants such as homologous (D614G) and heterologous strains such as B.1.128.2, B.1.351, B.1.1.7, B.617, B.617.2.

The said formulation is expected to be stable for at least 1-2 years at 2-8° C., 6 months at 25±2° C. and upto 14 days at 37±0.2° C.

The vaccine formulation of the present invention may be used for either prophylactic or therapeutic purposes.

Further, the present invention also discloses that dose sparing effect of inactivated Whole virion SARS-CoV-2 antigen, when formulated with Algel-IMDG® (BBV152A &B) compared to Algel (BBV152C).

The present invention also discloses that dose sparing effect of inactivated Japanese encephalitis antigen, when formulated with Algel-IMDG compared to antigen that was formulated with Algel.

Further, in another embodiment, present invention discloses the efficacy of an inactivated Whole virion SARS-CoV-2 adjuvanted vaccine formulations (BBV152A, BBV152B & BBV152C), demonstrated in Syrian hamster and non-human primate model, after the live virus challenge.

In another embodiment, present invention discloses the safety of an inactivated Whole virion SARS-CoV-2 adjuvanted vaccine formulations (BBV152 A, BBV152B & BBV152C).

Further, the present invention also discloses that SARS-CoV-2 adjuvanted Vaccine formulation (BBV152A & BBV152B) with this novel agonist-based adjuvant induces anti-viral cytokines, which is an indicative of activation of adaptive immunity.

Further, the present invention also discloses that adjuvanted Vaccine formulation with this novel agonist-based adjuvant induces CD4+ IFNγ T lymphocyte population. Anti-viral cytokines, which is an indicative of activation of adaptive immunity.

The present invention also discloses the cross-neutralization ability of sera, collected from the vaccinated human subjects with Inactivated SARS-CoV-2 vaccine formulated with Algel-IMDG (BBV152B).

### EXAMPLES

The above-described aspects of the invention further be understood by following non-limiting examples and corresponding drawing figures.

### Example 1: Preparation of (Algel-IMDG): Chemisorbed TLR7/8 Agonist Molecule on to the Surface of Algel

Algel-IMDG is m-Amine Gallamide N-(3-((4-amino-2-butyl-1H-imidazo[4,5-c]quinolin-1-yl) methyl) benzyl)-3,4,5-trihydroxybenzamide, a novel TLR7/8 agonist, chemisorbed on to the surface of aluminium hydroxide. This technology was Immidazoquinoline licensed from Virovax, USA.

### Example 1.1: Physico-Chemical Characteristics of IMDG

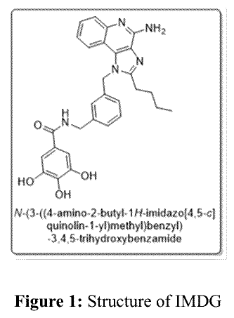

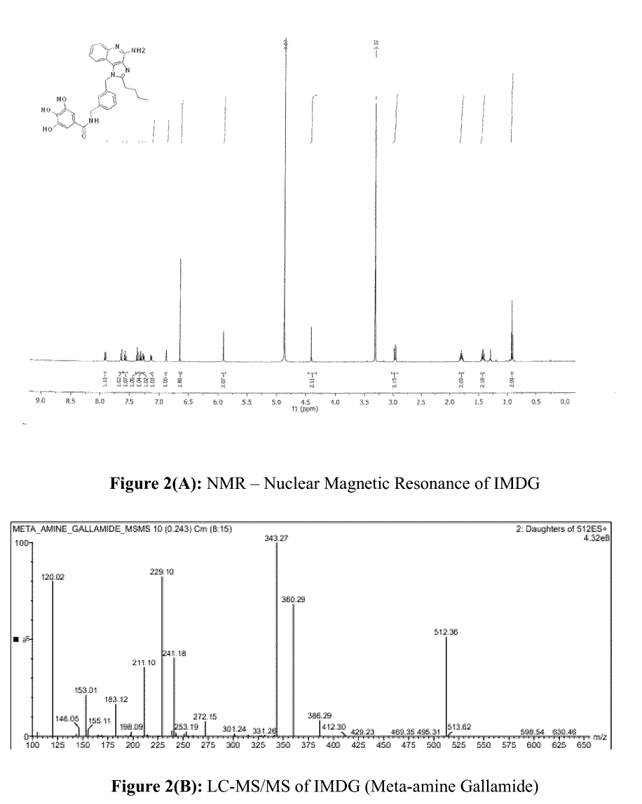

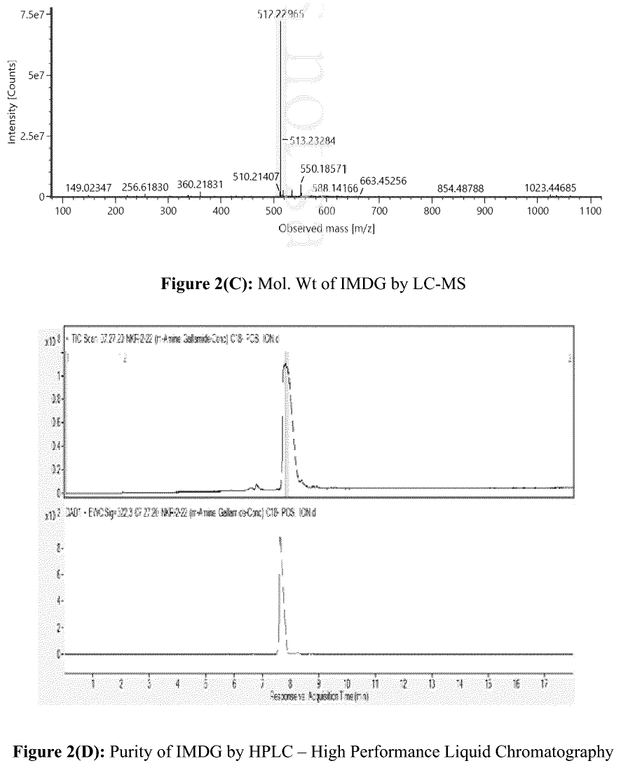

Meta amine Gallamide, namely Imidazoquinoline Gallamide (IMDG) (FIG. 1A), used for the preparation of Algel-IMDG was fully characterized by NMR & LC-MS/MS for Mass spec of IMDG is shown as in FIGS. 2A & 2B respectively and its molecular weight was also found to be 511+(as shown in Table 1 & FIG. 2C). It was further evaluated for the purity by HPLC using Photo diode Array (PDA) detector at a wavelength=322 nm and found to be >99% as shown in FIG. 2D.

TABLE 1

| | Component | Observed neutral | Neutral | Observed | Mass error | |
|---|---|---|---|---|---|---|
| S. No | name | mass (Da) | mass (Da) | m/z | (ppm) | Adducts |
| 1 | C29H29N5O4 | 511.2224 | 511.22195 | 512.2296 | 0.8 | +H |

### Example 1.2: ALGEL-IMDG Preparation

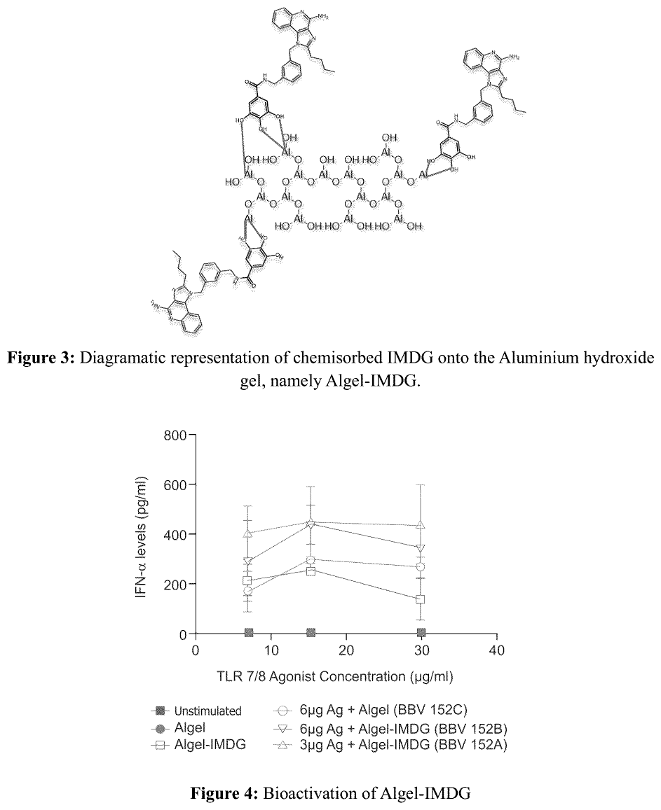

A total of 600-1000 mg of N-(3-((4-amino-2-butyl-1H-imidazo[4,5-c]quinolin-1-yl) methyl) benzyl)-3,4,5-tri-hydroxybenzamide was dissolved in 60-100 mL of 100% isopropanol. Solution was kept at 50° C. to dissolve completely and filtered using a 0.22-micron filter. AL hydrogel (1000-3000 mL) was aliquoted in sterile glassware and added the solution of N-(3-((4-amino-2-butyl-1H-imidazo[4,5-c]quinolin-1-yl) methyl) benzyl)-3,4,5-tri-hydroxybenzamide dropwise under continuous stirring, for 72 hours. FIG. 3 illustrates the structural relationship of IMDG and Aluminium hydroxide gel, namely Algel-IMDG.

### Example 1.3: Physico-Chemical Characteristics of ALGEL-IMDG

Physico-chemical characteristics of Algel-IMDG such as pH, viscosity and particle size were determined as per IH (International Harmonization) Specification, and the results were shown as in Table 2.

TABLE 2

| S. No | Description | Results |
|---|---|---|
| 1 | pH | 6.77 |
| 2 | Viscosity | 3.33 |
| 3 | Mol. Wt | 511+ |

1.3.1: IMDG quantification: Since, IMDG was aimed to chemisorb onto the aluminium hydroxide, (Algel-IMDG) was further subjected to LC-MS/MS to determine the quantity of bound and unbound IMDG. It was found that IMDG was strongly bound to Algel as determined by bound and unbound fractions by LC-MS. Negligible or undetectable IMDG was noticed in unbound fraction. More than 15 batches of Algel-IMDG were analysed and cumulative results of bound and unbound IMDG quantified by LC-MS is as represented in the Table 3 given below. Each analysis was run in triplicates.

TABLE 3

| | % IMDG Bound | % IMDG Unbound |
|---|---|---|
| Algel-IMDG | (Mean ± SD) | (Mean ± SD) |
| Cumulative data of 15 | 93 ± 13 | 0.214 ± 0.232 |
| batches | | |

1.3.2: Residual Isopropyl alcohol (IPA) of Algel-IMDG: Finally, prepared Algel-IMDG solution was checked for residual IPA content. To estimate the presence of residual Isopropyl alcohol (IPA), three batches of Algel-IMDG were analysed to estimate IPA by GC MS/MS. Sample analysis was done in triplicates. These results indicated the residual IPA is well within the approved permissible limits (5000 ppm), that is always <5000 ppm as shown in Table 4.

TABLE 4

Before Antigen addition

| S. No | Algel-IMDG Samples | Residual IPA (in PPM) |
|---|---|---|
| 1 | Sample 1 | 3642.78 |
| 2 | Sample 2 | 2241.84 |
| 3 | Sample 3 | 2122.64 |

1.3.3: Stability of Algel-IMDG: In order to address the stability of IMDG in Algel-IMDG adjuvant formulation, Algel-IMDG was stored at real time temperature (2-8° C.) and accelerated temperature (25° C.). A small aliquot was taken out at various time points and estimated both bound IMDG and Unbound IMDG by LC-MS. Based on the results obtained from the ongoing stability study as shown in Table 5, Algel-IMDG was found to be stable at 2-8° C. for upto 90 days, and accelerated temperatures such as at room temperature (25° C.) and 37° C., upto 15 days.

TABLE 5

| | 2-8° C. | | 25° C. | | 37° C. |
|---|---|---|---|---|---|
| Time | % Bound | Time | % Bound | Time | % Bound |
| Points | IMDG | Points | IMDG | Points | IMDG |
| Day 0 | 102.67 ± 9.34 | Day 0 | 92.39 ± 1.87 | Day 0 | 92.39 ± 1.87 |
| Day 10 | 116.59 ± 14.13 | Day 3 | 96.08 ± 2.39 | Day 3 | 93.69 ± 2.22 |
| Day 30 | 106.31 ± 16.43 | Day 7 | 95.2 ± 0.74 | Day 7 | 89.16 ± 1.03 |
| Day 60 | 101.49 ± 22.22 | Day 15 | 100.83 ± 1.91 | Day 15 | 92.43 ± 1.26 |
| Day 90 | 112.80 ± 17.36 | | | | |

### Example 2: Vaccine Formulation

To make, 1 ml of vaccine formulation, required antigen bulk volume containing (1-10 μg antigen) was taken and added 0.05 ml to 0.15 ml of Algel-IMDG ( 1/40^{th} dilution of Algel-IMDG) followed by the addition of preservative. The composition of vaccine formulation is as indicated in the table below (Table 6A & 6B)

TABLE 6A

| S. | | | |
|---|---|---|---|
| No | Vaccine Composition | Quantity | |
| 1 | Inactivated SARS-CoV-2 antigen | 6 | μg |
| 2 | Algel-IMDG | 250 or 500 or 750 μg Al³⁺ | |
| | | content with 15 or 25 μg IMDG | |
| 3 | 2-PE | 5 | mg/ml |

| | | |
|---|---|---|
| 4 | PBS | Qs to make 0.5 ml |

TABLE 6B

| S. | | |
|---|---|---|
| No | Vaccine Composition | Quantity |
| 1 | Inactivated Japanese Encephalitis | 3 or 6 μg |
| | antigen | |
| 2 | Algel-IMDG | 250 or 500 or 750 μg Al³⁺ |
| | | content with 15 or 25 μg IMDG |
| 3 | 2-PE | 5 mg/ml |
| 4 | PBS | Qs to make 0.5 ml |

### Example 2.1: Vaccine Formulation with Varying Concentrations of Al³⁺ of Aluminium Hydroxide Gel

2.1.1: Immunization: BALB/c were immunized with PBS or adjuvanted vaccine formulations containing whole virion inactivated SARS-CoV-2 antigen with Algel-IMDG, wherein Algel-IMDG was formulated with either with 1 or 0.75 or 0.5 mg of Al³⁺ content/ml. These formulations were administered via IM with 1/10^{th} human single dose on day 0 and 14. Blood was collected from all animals on Day 14 and Day 28, before the vaccination. Sera was separated and stored at −20° C. until further use.

2.1.2: ELISA: Spike (S1) specific antibodies were determined by ELISA and found that all three groups elicits high antibody binding titers and the titers are found to be similar without any statistical significant difference. However, group of animals that received Algel-IMDG with 1 mg/ml of Al³⁺ concentration showed less titer compared to other two groups that received Algel-IMDG with 0.75 & 0.5 mg/ml of Al³⁺ concentration respectively (Table 7A). Table 7A, indicates Geometric mean titers obtained against three formulations in mice and Table 7B indicates antibody isotyping.

TABLE 7A
Spike (S1) specific End point Antibody
Binding Titer, (GMTs, at 95% CI)

Algel-IMDG
(Al³⁺ Concentration in mg/ml)

| PBS | 1 mg/ml Al³⁺ | 0.75 mg/ml Al³⁺ | 0.5 mg/ml Al³⁺ |
|---|---|---|---|
| 50 | 204800 | 1158524 | 819200 |

TABLE 7B

| Algel-IMDG | Th1:Th2 Index |
|---|---|
| (Al³⁺ Concentration | (Ratio of IgG2a/IgG1 Ab titers) |
| in mg/ml) | (GMTs, at 95% CI)* |
| 1 | 0.794 |
| 0.75 | 0.574 |
| 0.5 | 4.0 |

Algel-IMDG with 0.5 mg/ml of Al³⁺ concentration showed Th1 biased response than the other two formulations containing 1 mg & 0.75 mg/ml of Al³⁺ concentration, which showed balanced immune response more towards Th2 rather than Th1 response. Hence, the Algel-IMDG with 0.5 mg/ml of Al³⁺ concentration is optimal, which further induces Th1 biased immune response required to provide long term immunity against SARS-CoV-2.

### Example 2.2: Physico-Chemical Characteristics of Vaccine Formulation

2.2.1: Antigen adsorption: Percent antigen adsorption to Algel-IMDG was also estimated by Lowry method, after desorption. The results were found to be >90%.

2.2.2: IMDG concentration in the final vaccine formulation: IMDG concentration in the finished product or vaccine formulation was also subjected to LC-MS/MS to determine the quantity of IMDG. Vaccine formulations were analysed and cumulative results of total IMDG, quantified by LC-MS is as represented in the Table 8 given below. Each analysis was run in triplicates.

TABLE 8

Vaccine formulation with Algel-IMDG
against Viral infections

| | Inactivated | |
|---|---|---|
| | SARS-CoV-2* | Inactivated JE# |
| | (Target IMDG | (Target IMDG |
| IMDG in Vaccine | Concentration, | Concentration, |
| formulation | 30 μg/ml) | 50 μg/ml) |
| Total IMDG estimated | 30.82 ± 3.55 | 53.89 ± 7.17 |
| (Mean ± SD) | | |
| % IMDG estimated | 102.74 ± 11.82 | 107.78 ± 14.34 |
| (Mean ± SD) | | |

NOTE:
*15 batches were analysed;
#3 samples were analysed.

2.2.3: Residual Isopropyl alcohol (IPA) of final vaccine formulation: Final, prepared vaccine formulation was checked for residual IPA content. To estimate the presence of residual Isopropyl alcohol (IPA), three batches of finished product or vaccine formulation were analysed to estimate IPA by GC MS/MS. Sample analysis was done in triplicates. These results indicated the residual IPA is well within the approved permissible limits (5000 ppm), that is always <5000 ppm as shown in Table 9.

TABLE 9

After Antigen Addition

| | Finished product | Residual IPA |
|---|---|---|
| S. No | (BBV152B) | (in PPM) |
| 1 | Sample 1 | 99.09 |
| 2 | Sample 2 | 98.61 |
| 3 | Sample 3 | 24.42 |

2.2.4: Stability of Vaccine (BBV152B): In order to address the stability of Vaccine formulation, prepared using Algel-IMDG, vaccine was stored at real time temperature (2-8° C.) accelerated temperature (25° C.) and 37° C. temperature. A small aliquot was taken out at various time points as indicated in Table 10 and estimated total protein by Lowry method. Based on the results obtained from the ongoing stability study, protein is stable upto 12 months at 2-8° C., 6 months at 25±2° C. and upto 14 days at 37±0.2° C.

TABLE 10

| Test parameter | Time point | 2-8° C. | Time point | 25 ± 2° C. | Time point | 37 ± 0.2° C. |
|---|---|---|---|---|---|---|
| Total protein | 3 months | 7.9 μg/ | 1 month | 6.3 μg/ | 2 days | 7.8 μg/ |
| (μg/0.5 ml) | | 0.5 ml | | 0.5 ml | | 0.5 ml |
| Total protein | 6 months | 6.7 μg/ | 3 months | 6.2 μg/ | 7 days | 5.3 μg/ |
| (μg/0.5 ml) | | 0.5 ml | | 0.5 ml | | 0.5 ml |
| Total protein | 9 months | 7 μg/ | 6 months | 6.7 μg/ | 14 days | 6.2 μg/ |
| (μg/0.5 ml) | | 0.5 ml | | 0.5 ml | | 0.5 ml |
| Total protein | 12 months | 6.4 μg/ | | | | |
| (μg/0.5 ml) | | 0.5 ml | | | | |

### Example 3: Bioactivity of Adjuvanted Vaccine Formulation with Inactivated SARS-CoV-2 Antigen

Human PBMCs (Peripheral blood mononuclear cells) collected from normal healthy individuals were resuspended in complete media i.e RPMI 1640 with 10% FBS and supplemented with Penicillin/Streptomycin/Glutamine media. PBMCs were plated in 96 well plate (50,000×10⁶/well) in triplicates and stimulated with 5-fold serial dilutions of Inactivated whole virion SARS-CoV-2 antigen (3 μg & 6 μg) and Adjuvanted vaccine formulations (6 μg Ag with Algel and 3 μg & 6 μg Ag with novel adjuvant). Cells were incubated in a humidified 5% CO₂ environment for 48 hrs. Supernatant collected was used to estimate IFN-α. PBMCS stimulated with SARS-CoV-2 Adjuvanted vaccine formulations showed the induction of Interferon alpha, a general inflammatory cytokine which skews the immune response towards a Th1 profile (FIG. 4).

### Example 4: Dose Sparing Effect of Adjuvanted Vaccine Formulation

### Example 4.1: Dose Sparing Effect of Adjuvanted Vaccine Formulation with Inactivated Japanese Encephalitis Antigen

4.1.1: Immunization: In this example, mice were vaccinated to evaluate the immunogenicity of an adjuvanted vaccine formulations (at 3 g & 6 g antigen concentration). For this, Balb/C mice (n=6) were injected via intraperitoneal route with 1/20^{th} dilution of adjuvanted vaccine formulations containing 3 g & 6 g antigen concentration 0.5 ml/mouse) on day 0 and 7. Sera was separated and used to evaluate neutralization antibody titer by PRNT₅₀. Addition of novel adjuvant showed 2-fold dose sparing effect by showing the similar titer as that of 6 mcg formulation (Table 11).

TABLE 11
Dose sparing effect of Novel Adjuvant in
Inactivated Japanese Encephalitis antigen

| Group No. | Description | Antibody Titer |
|---|---|---|
| 1 | PBS Control | 20 |
| 2 | Algel | 126.89 |
| 3 | Algel-IMDG | 177.11 |
| 4 | JE Concentration 1 (6 μg) | 84.52 |
| 5 | JE Concentration 2 (3 μg) | 56.29 |
| 6 | JE Concentration 1 (6 μg) + Algel | 325.81 |
| 7 | JE Concentration 2 (3 μg) + Algel | 246.96 |
| 8 | JE Concentration 1 (6 μg) + Algel-IMDG | 868.04 |
| 9 | JE Concentration 2 (3 μg) + Algel-IMDG | 2680.99 |

Note
Wherever used in the description, Algel-IMDG represents novel adjuvant of the invention.

### Example 4.2: Dose Sparing Effect of Adjuvanted Vaccine Formulation with Inactivated SARS-CoV-2 Antigen

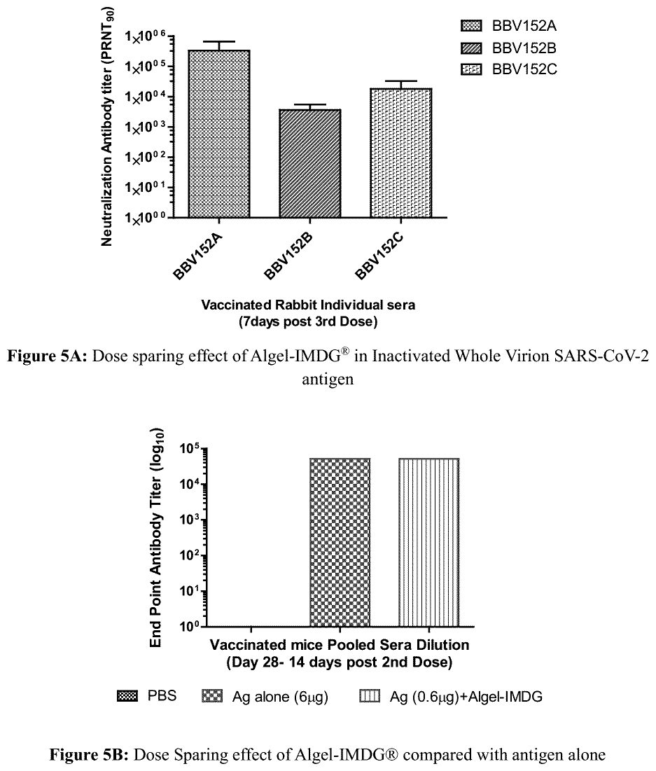

4.2.1: Immunization: New Zealand white rabbits (3-4 months old) were vaccinated via intramuscular route with full Human intended single dose (HSD, 3 μg or 6 μg) of inactivated whole virion vaccine with Algel (6 μg Ag, BBV152C) or novel adjuvant (Algl-IMDG) (3 μg Ag-BBV152A & 6 μg Ag-BBV152B) on days 0, 7 & 14 days (N+1 dose). Sera was separated and used to evaluate neutralization antibody titer by PRNT₉₀. Adjuvanted formulation with novel adjuvant (Algel-IMDG) showed dose sparing effect compared to Adjuvanted formulation with Algel, by at least 2-fold (FIG. 5A).

4.2.2: Immunization: Balb/C mice (n=6) were injected via intramuscular route with 6 μg antigen and Adjuvanted vaccine formulation (0.6 μg Ag with Algel-IMDG/0.1/mouse) on day 0, and 14. Sera was separated and used to evaluate the antigen specific antibody titers by ELISA.

Adjuvanted Vaccine formulation with Algl-IMDG showed 10 times more dose sparing effect compared to antigen alone (FIG. 5B).

### Example 5: Induction of Th1 Biased Immune Response

### Example 5.1: Induction of Th1 Biased Immune Response in BALB/c Mice

5.1.1: Animals: Balb/C (6-8 weeks old, female) mice were purchased and maintained in the animal care facility under standard approved protocols. All procedures involving mice were carried out with the approval of Institutional Animal Ethics Committee.

5.1.2: Immunization: In this example, mice were vaccinated to evaluate the immunogenicity of an adjuvanted vaccine formulation. For this, Balb/C mice (n=6) were injected via intraperitoneal route with 1/10^{th} HSD (3 μg & 6 μg/0.5 ml/mouse) on day 0, 7 and 14. Sera was separated and used to evaluate the antigen specific antibody titers & its antibody isotypes (IgG₁, IgG₂ₐ or IgG₃) by ELISA. Absorbance was read at 450 nm. Threshold (Mean+3SD) was established by taking the absorbance of negative control (PBS) group.

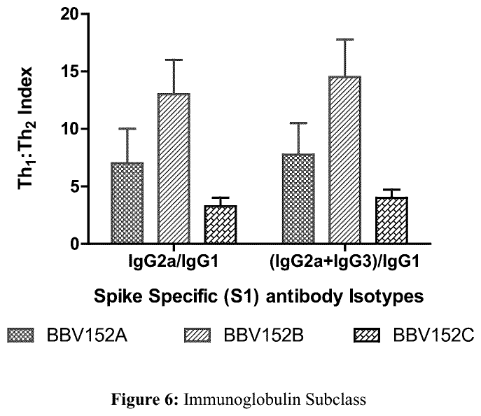

Sera samples collected on Day 21 from vaccinated mice were also analyzed for antibody isotypes by indirect ELISA using Goat anti-mouse IgG1 or IgG2a HRP Conjugate antibody. Results showed that there is a distinct Th1 Biased immune response elicited against either antigen alone and adjuvanted vaccine formulations (Algel and Algl-IMDG), which was calculated based on Th1/Th2 ratio, based on the end point titer of IgG2a vs IgG1 (FIG. 6).

5.1.3: Ab isotyping: Immunoglobulin subclasses (IgG1, IgG2a and IgG3) were analyzed on day 14 hyperimmunized BALB/c mouse sera samples to evaluate the Th1/Th2 polarization. The average ratio of IgG2a/IgG1 or IgG2a+IgG3/IgG1 was higher in Algel-IMDG groups when compared to Algel, indicative of Th1 bias (FIG. 6). Antigen alone showed Th1 biased response at three tested different concentrations with an average Th1.Th2 index of 3, however, ELISA & PRNT₉₀ titers are less compared to Adjuvanted vaccine formulations.

### Example 5.2: Induction of Th1 Biased Immune Response in Human PBMCs

In a double-blind, multicentre, randomised, controlled phase 1 trial, safety and immunogenicity of BBV152 was assessed in healthy adults aged 18-55 years. In this trial, Individuals with positive SARS-CoV-2 nucleic acid and/or serology tests were excluded. Participants were randomly assigned to receive either one of three vaccine formulations (3 μg with Algel-IMDG, 6 μg with Algel-IMDG, or 6 μg with Algel) or an Algel only control vaccine group. Block randomisation was done with a web response platform. Participants and investigators were masked to treatment group allocation. Two intramuscular doses of vaccines were administered on day 0 (the day of randomisation) and day 14. Day 0 & Day 28 human PBMCs were analysed for cell mediated responses by ELISpot assay and intracellular staining.

5.2.1: Induction of Th1 biased Immune Response in human PBMCs by ELISpot: Cell-mediated responses were assessed in a subset of participants at one site (NIMS). Blood (3-5 mL) was collected from those participants who gave consent to give additional volume of blood on days 0 and 28.

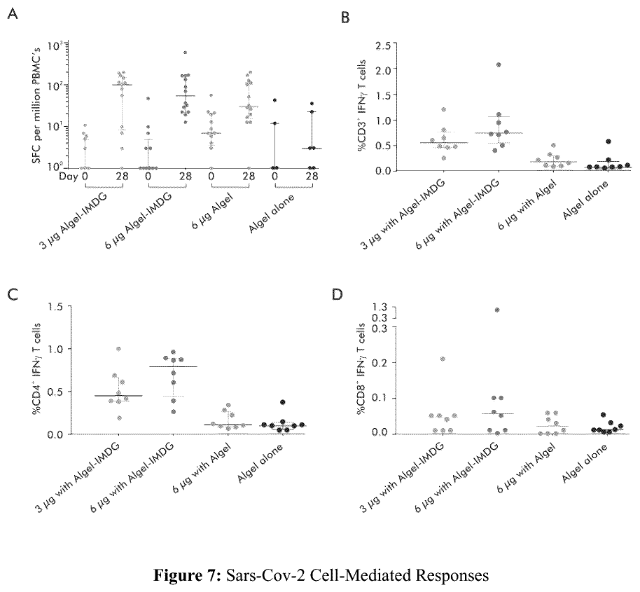

ELISpot Assay: Peripheral blood mononuclear cells were collected to assess IFN-γ by ELISpot (13 in vaccinated groups and six in the control group) and performed as per the manufacturer's instructions (MABTECH). Briefly, ELISPOT plates precoated with IFN-γ antibody were used, these were further seeded with 300,000 PBMCs obtained from the study subjects. The PBMCs were stimulated with SARS-CoV-2 peptide matrix (SARS-CoV-2 S1 scanning pool) (MABTECH) at a concentration of 5 ug/ml for 18 hours. Unstimulated cells and anti-CD3 stimulated cells were used as a negative and positive controls, respectively. Subsequently, the plates were washed and incubated with a biotinylated detection antibody, followed by Streptavidin-ALP (Alkaline Phosphatase). The plates were developed with the BCIP-NBT substrate (5-bromo-4-chloro-3′-indolyphosphate and nitro-blue tetrazolium) as per the manufacturer's instructions until distinct spots emerged. The number of blue spots per well was determined by using an ELISPOT reader (AiD) or under a dissection microscope (Leica). The frequency of positive cells was calculated after subtracting the number of spots in unstimulated cells from the peptide stimulated cells, and the results were expressed as SFU/106 PBMCs. IFN-γ ELISpot responses against SARS-CoV-2 peptides peaked at about 100-120 spot-forming cells per million peripheral blood mononuclear cells in all vaccinated groups on day 28 (FIG. 7A), that means vaccine formulated with Algel or Algel-IMDG.

5.2.2: Induction of Th1 biased Immune Response in human PBMCs by intracellular staining: Intracellular cytokine staining was used to assess T-cell responses in the remaining samples that contained an adequate number of cells. To ensure equal distribution, eight samples in each vaccine group were selected. All samples were analysed for intracellular staining.

Intracellular Staining: Human PBMCs (1×10⁶/ml) were cultured in 24 well plates and stimulated with inactivated SARS-COV-2 antigen (1.2 μg/ml) or PMA (25 ng/ml, cat #P8139; Sigma) and Ionomycin (1 μg/ml, cat #10634, Sigma) along with Protein transport inhibitor (Monensin, 1.3 μl/ml cat #554724, BD biosciences) for 12-16 hrs in C02 incubator at 37° C. Cells were washed and centrifuged at 1000 rpm for 5-10 min and stained with cell surface markers BV421 Mouse Anti Human CD3 (clone: UCHT1, Cat #562427, BD Biosciences), APC-Cy7 Mouse Anti Human CD4 (Clone:SK3 Cat #566319, BD Biosciences) and PE-Mouse Anti Human CD8a (Clone: HIT8a, Cat #555635, BD Biosciences) for 30 minutes at 4° C. Cells were again washed twice with PBS and fixed using fixation/Permeabilize solution (Cat #554722, BD Biosciences) for 20 mins at 4° C. Following fixation/permeabilization, cells were washed with 1×permeabilization buffer and stained with intracellular cytokines IFN-γ (APC Mouse Anti Human IFN-7, Clone: 4S. B3, cat #551385, BD Biosciences) for 30 mins at 4° C. Cells were washed and resuspended in 500 μl FACS buffer (Cat #554657, BD Biosciences). All samples were acquired using BD FACSVerse (BD Biosciences).

Each assay was performed while maintaining positive and negative controls. Cells stimulated with PMA, ionomycin used as a positive control. Unstimulated cells, PBMCs from unvaccinated individuals or PBMCs collected on Day 0 from vaccinated individuals used as a negative control.

CD4+ and CD8+ T-cell responses were detected in a subset of 16 participants from both Algel-IMDG groups. Both the Algel-IMDG groups elicited CD3+, CD4+, and CD8+ T-cell responses that were reflected in the IFN-γ production. However, there was a minimal detection of less than 0·5% of CD3+, CD4+, and CD8+ T-cell responses in the 6 μg with Algel group and the Algel only group. (FIGS. 7B, C & D)

### Example 6: Evaluation of Efficacy of Vaccine Formulation, after Wild Type (NIV-NIV-2020-770 with a Mutation at D614G) Live Virus Challenge

To assess the immunogenicity and protective efficacy of inactivated SARS-CoV-2 vaccine candidates BBV152A, BBV152B, and BBV152C were used to vaccinate both syrian hamster and NHP model with three and two dose vaccination regimen respectively, followed by live virus challenge after the last dose.

### Example 6.1: Syrian Hamster Model

6.1.1: Immunization: Thirty-six, 6-8 weeks old female Syrian hamsters were divided into four groups, viz., Group I, II, III and IV of 9 hamsters each. The hamsters were housed in individually ventilated cages with ad libitum food and water. Group I was administered with phosphate-buffered saline (PBS), group II with BBV152C (6 g of vaccine candidate along with Algel), group III with BBV152A (3 μg of vaccine candidate with Algel-IMDG) and group IV with BBV152B (3 g of vaccine candidate with Algel-IMDG). Animals of each group were immunized with 0.1 ml of PBS/vaccine formulations intramuscularly in the hind leg under isoflurane anaesthesia on 0, 14 and 35 days. The hamsters were bled on days 12, 21 and 48 post-immunization to check for antibody response.

6.1.2: Wild type (NIV-NIV-2020-770 with a mutation at D614G) Live virus Challenge studies: The immunized hamsters were challenged with 0.1 ml of 10^{5.5} TCID50 SARS-CoV-2 virus intranasally on the eighth-week post-immunization (day 50) in the containment facility of ICMR-National Institute of Virology, Pune under isoflurane anaesthesia. Throat swabs were collected in 1 ml virus transport media on every alternate day post inoculation for viral load estimation. Three hamsters from each group were euthanized on 3, 7 and 15 DPI to collect throat swab, nasal wash, rectal swab, blood and organ samples for viral RNA estimation, titration, histopathology, and immunological analysis.

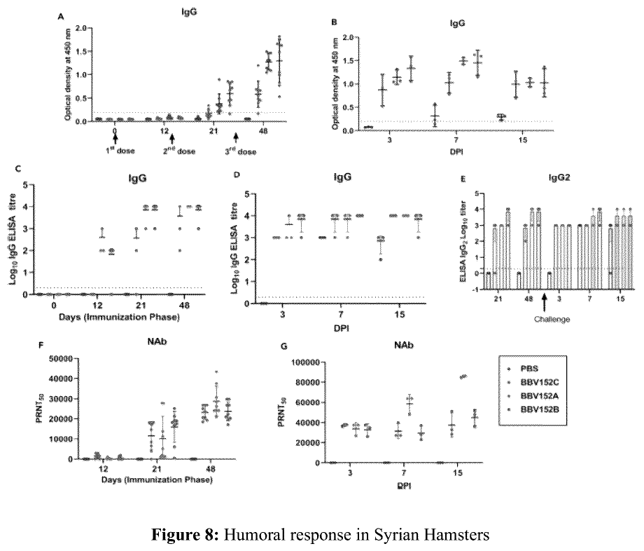

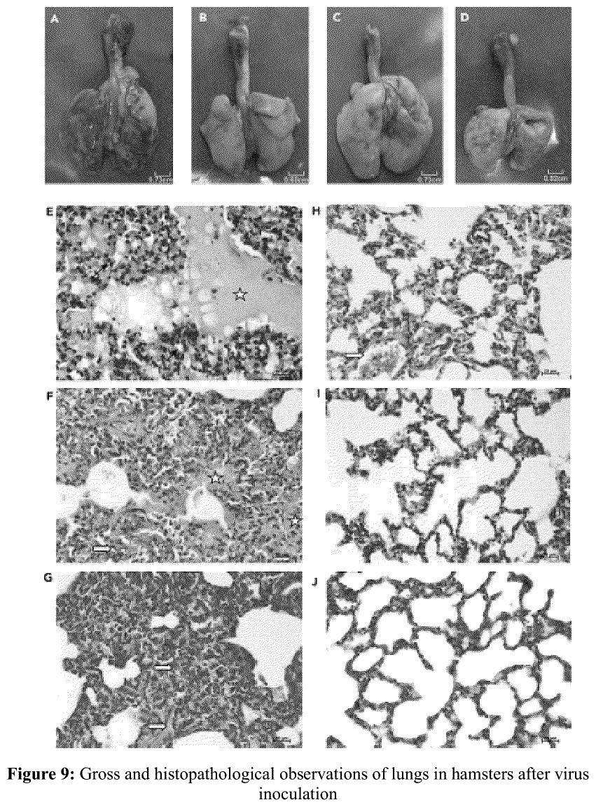

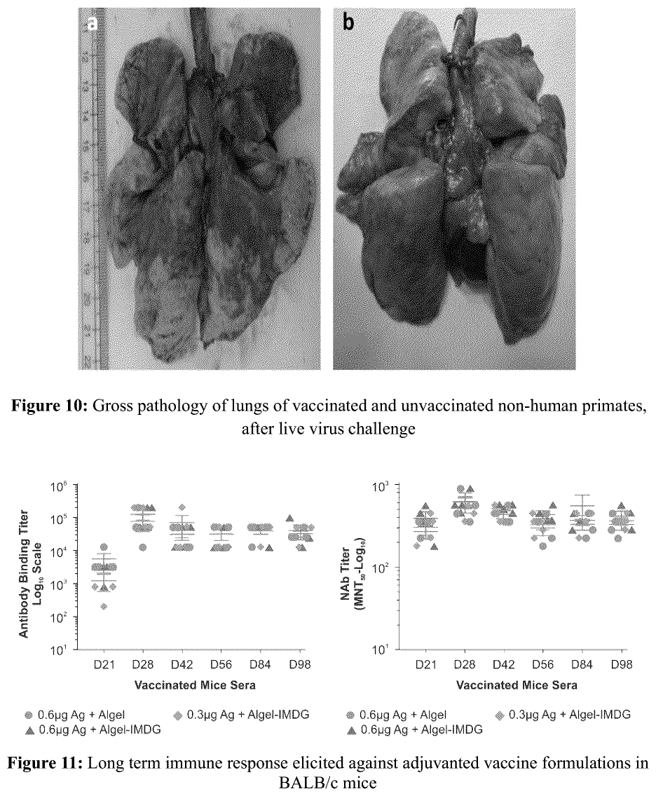

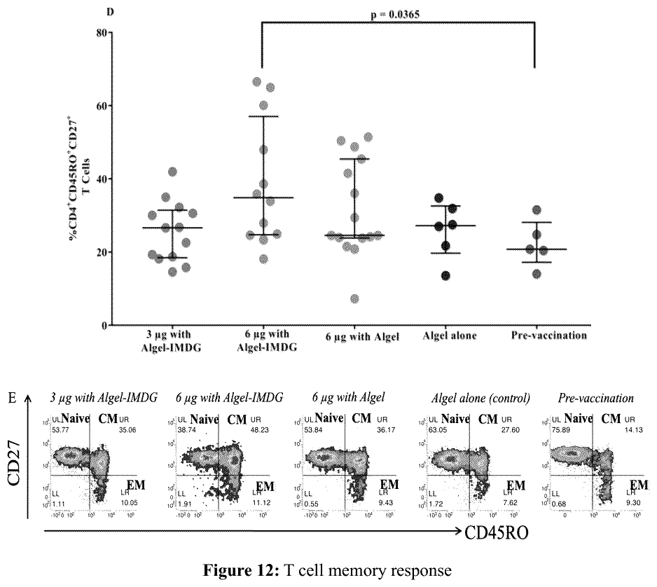

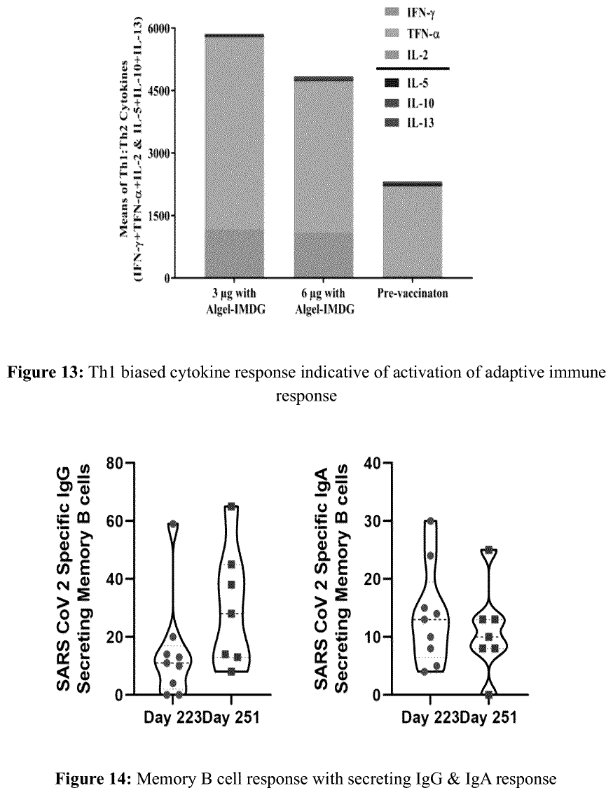

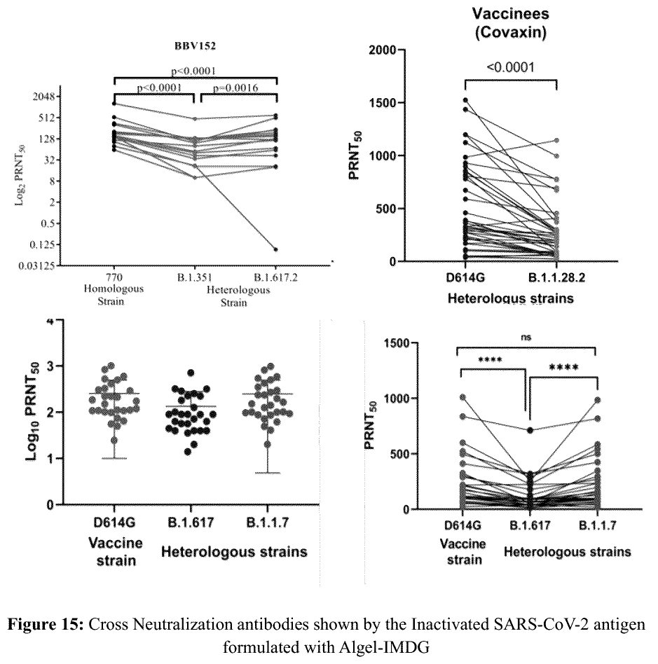

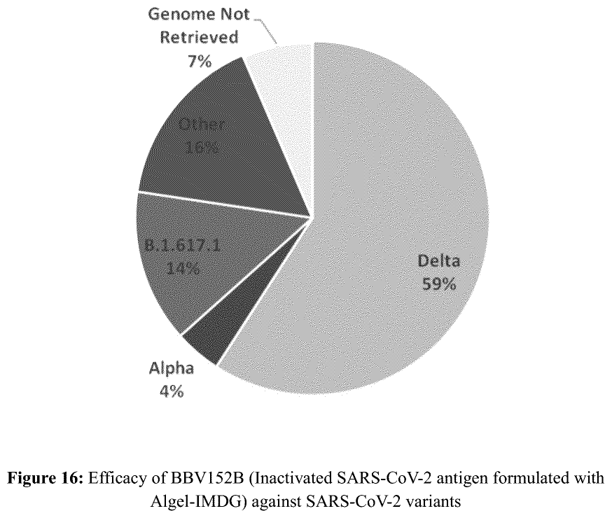

All vaccine candidates induced significant titers of SARS-CoV-2-specific IgG and neutralizing antibodies. BBV152A and BBV152B vaccine candidates remarkably generated a quick and robust immune response (FIGS. 8A, B, C, D, E, F & G). Post-SARS-CoV-2 infection, vaccinated hamsters did not show any histopathological changes in the lungs. The protection of the hamster was evident by the rapid clearance of the virus from lower respiratory tract, reduced virus load in upper respiratory tract, absence of lung pathology, and robust humoral immune response. These findings confirm the vaccine composition prepared with Algel-IMDG showed better protection of hamsters challenged with SARS-CoV-2 (FIGS. 9A to J).

### Example 6.2: Non-Human Primate Model

Protective efficacy and immunogenicity of an inactivated SARS-CoV-2 vaccine was also evaluated in rhesus macaques.

6.2.1: Immunization: Twenty macaques were divided into four groups of five animals each. One group was administered a placebo, while three groups were immunized with three different vaccine candidates of BBV152 at 0 and 14 days. All the macaques were challenged with SARS-CoV-2 fourteen days after the second dose.

The protective response was observed with increasing SARS-CoV-2 specific IgG and neutralizing antibody titers from 3rd-week post-immunization. Viral clearance was observed from bronchoalveolar lavage fluid, nasal swab, throat swab and lung tissues at 7 days post-infection in the vaccinated groups. No evidence of pneumonia was observed by histopathological examination in vaccinated groups, unlike the placebo group which exhibited interstitial pneumonia and localization of viral antigen in the alveolar epithelium and macrophages by immunohistochemistry. The gross pathology of the lungs of animals of the placebo group at 7 DPI showed a significantly higher incidence of bronchopneumonic patches and consolidation at necropsy, as compared to the vaccinated groups (FIG. 10). Further, there were no signs of eosinophilic infiltration in the lungs in the histopathology on the day of sacrifice, indicative of no association with vaccine-enhanced disease.

### Example 7: Long Term Immunogenicity

Mice: To demonstrate long-lived immune response, BALB/c mice (n=8/group, 4 male and 4 female) were vaccinated intramuscularly with three adjuvanted vaccine formulations (1/10th HSD of BBV152A, B, and C) on day 0, 7, and 14 and evaluated antibody titer up to 12 weeks after last dose. These results revealed that the spike-specific antibodies reached peak level on 10 day 28, and the antibody titers were sustained up to day 98, i.e. 12 weeks after last dose (FIG. 11A). Similarly, we also found sustained NAb titers up to day 98 (FIG. 11B), which indicates the BBV152 vaccine candidates were able to produce long-term immunity.

### Example 8: Safety of Algel-IMDG & Adjuvanted Vaccine Formulation

Extensive safety evaluation was performed for the Algel-IMDG adjuvant alone as per the regulatory guidelines (WHO, 2013; OECD, 2020; CDSCO, 2019). Tests such as (i) Mutagenicity assay (in-vitro) to determine mutagenic potential of the Algel-IMDG, (ii) Maximum Tolerated Dose test (MTD, in-vivo), to ensure the human intended adjuvant dose is tolerable, and (iii) Repeated Dose toxicity study (RDT, In-vivo), to evaluate that repeated administration of Algel-IMDG does not cause any systemic toxicity or mortality.

### Example 8.1: Safety of Algel-IMDG Alone

8.1.1: Mutagenicity assay performed with Algel-IMDG at various concentrations revealed no substantial increase in revertant colony numbers in any of the tested strains at different dose level, in both the plate-incorporation and pre-incubation methods in the presence or absence of metabolic activation (S9 mix). Thus, the Algel-IMDG used in the formulation of BBV152 was found to be non-mutagenic.

8.1.2: Single Dose Toxicity of Algel-IMDG: Maximum tolerated dose study performed with single dose of Algel-IMDG also revealed that the Algel-IMDG was tolerated at the tested dose (20 μg agonist/animal) in Swiss Albino mice and Wistar rats as demonstrated by lack of erythema, edema, or any other macroscopic lesions at the site of injection. However, local reaction was observed microscopically.

8.1.3: Repeated dose toxicity study (RDT, in-vivo) of Algel-IMDG: Repeated administration of (N+1 dose regimen) high dose of Algel-IMDG alone (30 μg agonist/animal) was performed in Swiss Albino mice. These results did not show any clinical signs, change in body weight, or histopathological changes, except local reaction at the site of injection and thus established the safety of Algel-IMDG at high dose.

### Example 8.2: Repeated Dose Toxicity Study (RDT, In-Vivo) of Adjuvanted Vaccine Formulations (BBV152 A, B & C)

Repeated administration of (N+1 dose regimen) high dose of adjuvanted vaccine formulation (9 μg Ag with 30 μg agonist/animal, which is more than HSD) in Wistar rats did not show any clinical illness, change in body weight, or histopathological changes, except inflammation at the site of injection and thus established the safety of both Algel-IMDG and adjuvanted vaccine formulations at high dose.

All three vaccine formulations (BBV152 A, B & C) were tested in three animal models (BALB/c mice, S. albino mice, and NZW rabbits) by the repeated dose toxicity study. These results demonstrated that vaccine formulations prepared with Algel-IMDG or Algel found to be safe, no mortality and with no changes in clinical signs, body weight gain, body temperature, or feed consumption in any of the animals.

Clinical pathological parameters such as haematology, clinical biochemistry, coagulation studies, and urinalysis performed in repeated dose toxicity (RDT) studies, showed that the animals administered either with adjuvanted vaccine candidates or adjuvants/antigen-alone were comparable to control, except increased levels of Alpha 1-acid glycoprotein and neutrophils count on Day 2 in adjuvant-alone or adjuvanted vaccine formulation groups.

However, these values were comparable to control on Day 21. This transient increase may be due to inflammation at the injection site after administration of the first dose. These findings were further correlated with the inflammatory reaction at the injection site observed microscopically, in the animals administered with adjuvant-alone and adjuvanted vaccine with Algel and Algel-IMDG. This inflammation was found to be slightly higher in animals that received Algel-IMDG than in animals which received Algel. However, this inflammation reduced by day 28. Other than local reaction at the site of injection, no other treatment-related microscopic findings observed in any of the animals administered with antigen or adjuvant or adjuvanted vaccine formulations. Histopathological examination of organs such as spleen, lungs, heart and lymph nodes etc. of all animal models administered with antigen or adjuvant or adjuvanted vaccine formulations were normal.

### Example 8.3: Safety of Adjuvanted Vaccine Formulations in Humans

Human Clinical trials (Phase 1) have been initiated using the whole virion inactivated vaccine formulations to assess the safety and immunogenicity of BBV152 at 11 hospitals across India.

8.3.1: Study Design: In this study, Healthy adults aged 18-55 years who were deemed healthy by the investigator were eligible. Individuals with positive SARS-CoV-2 nucleic acid and/or serology tests were excluded. Participants were randomly assigned to receive either one of three vaccine formulations (3 g with Algel-IMDG, 6 g with Algel-IMDG, or 6 g with Algel) or an Algel only control vaccine group. Block randomisation was done with a web response platform. Participants and investigators were masked to treatment group allocation. Two intramuscular doses of vaccines were administered on day 0 (the day of randomisation) and day 14. Primary outcomes were solicited local and systemic reactogenicity events at 2 h and 7 days after vaccination and throughout the full study duration, including serious adverse events.

All Adjuvanted vaccine formulations with Algel-IMDG (BBV152A, BBV152B) & with Algel (BBV152C) have been tested in 375 healthy human subjects, under the registered clinical trial (NCT04471519) at ClinicalTrials.gov. All the formulations found to be safe. Solicited local and systemic adverse reactions were reported by 17 (17%; 95% CI 10·5-26·1) participants in the 3 μg with Algel-IMDG group, 21 (21%; 13·8-30·5) in the 6 g with Algel-IMDG group, 14 (14%; 8·1-22·7) in the 6 μg with Algel group, and 10 (10%; 6·9-23·6) in the Algel-only group. The most common solicited adverse events were injection site pain (17 [5%] of 375 participants), headache (13 [3%]), fatigue (11 [3%]), fever (9 [2%]), and nausea or vomiting (7 [2%]). All solicited adverse events were mild (43 [69%] of 62) or moderate (19 [31%]) and were more frequent after the first dose. Hence, BBV152 with Algel-IMDG formulations led to tolerable safety outcomes.

### Example 9: Activation of Adaptive Immune Response and its Th1 Biased Immune Response

Cell-mediated responses were assessed in a subset of participants at three sites on day 42. The contract research organisation generated a random code containing randomisation numbers, which was provided to the staff to identify this subset of participants. Blood (3-5 mL) was collected on day 42 from 58 participants (29 from each group), who gave consent to collect additional volume of blood. Peripheral blood mononuclear cells (PBMCs) were separated and used to assess Th1 and Th2 cytokines. Ten pre-vaccination samples (five from each group) collected on day 0 served as the negative control. Th1 mediated cytokines (interferon-γ [IFNγ], tumour necrosis factor-α [TNFα], and IL-2) and Th2 mediated cytokines (IL-5, IL-10, and IL-13) were measured using a Luminex multiplex assay (Luminex Corporation, Austin, TX, USA) at Indoor Biotechnologies (Bangalore, India). (FIG. 13)

### Example 10: T Cell Memory Response

The generation of effective and persistent T cell memory is essential for long-term protective immunity to the virus. Especially, while developing vaccine against SARS-CoV-2, T cell response has become key determinant to assess the effectiveness of the vaccine. Hence, the ability to generate potentially protective response against SARS-CoV-2 infection, will determine the fate of the vaccine. The present study focussed to understand generation of B and T cell memory responses in the vaccinated individuals.

PBMCs from a subset of randomly selected participants who consented to the additional blood volume were collected on day 104 of the phase 1 trial and used to assess T-cell memory responses (CD4+CD45RO+ T cells and CD4+CD45RO+CD27+ T cells).

PBMCs from a subset of phase 1 participants at one site were collected to evaluate T-cell memory responses at day 104. Formulations with Algel-IMDG generated a T-cell memory response, as shown by an increase in the frequency of effector memory CD4+CD45RO+ T cells and CD4+CD45RO+CD27+ T cells compared with pre-vaccination (day 0) samples (FIG. 12).

### Example 11: B Cell Memory Response after 6 Months of 2^{Nd} Dose

Memory B cells are an important component of humoral immunity and contribute to viral control by generating antibody responses upon re-exposure to the virus or pathogen, which is further indicative of the presence of long-term immunity. To confirm, whether the BBV152B formulation able to generate antibody secreting B cell, also known as memory B cell, we performed B cell memory ELISpot assay.

To perform this assay, PBMCs were collected from a subset of individuals, who participated in Phase II clinical trial were analysed for B cell memory phenotype. The results obtained indicated that BBV152B formulation generates B cells that secretes IgG or IgA as shown in FIG. 14, upon antigen re-exposure. This confirms that BBV152B formulation able to induce long term immunity.

### Example 12: Cross Neutralization with Other SARS-CoV-2 Variants

Sera collected (4 weeks after the second dose) from 38 vaccine recipients, who received the BBV152 vaccine candidate in Phase II trial were subjected to PRNT₅₀ assay to underline the immunogenicity of the BBV152 vaccine candidate against SARS-CoV-2 UK variant with (VOC) 202 012/01 hallmarks belonging to GR clade and strain hCoV-19/India/2020/770 belonging to G clade. No significant reduction in neutralization of any SARS-CoV-2 variant of concern by sera of recipients who received BBV152 A or B.

Hence, it is concluded that sera from the BBV152A or BBV152B vaccine recipients did neutralize both homologous (D614G) and heterologous SARS-CoV-2 strains such as B.1.128.2, B.1.351, B.1.1.7, B.617, B.617.2. as shown in FIG. 15.

### Example 13: Efficacy of BBV2B Vaccine Candidate Against Sars-CoV-2 Infections

The Phase III clinical trial (#NCT04641481E) conducted to evaluate the efficacy of BBV152B vaccine was also shown or better protection against other circulating SARS-CoV-2 variants (FIG. 16). Among the 16,973 participants in the per protocol analysis population, there were 24 (0·28%) cases among 8471 participants in the vaccine arm and 106 (1·25%) cases among 8502 participants in the placebo group, resulting in estimated vaccine efficacy of 77·8% (95% CI: 65·2-86·4). There were sixteen cases who met the severe symptomatic COVID-19 cases definition, all but one of whom were in the placebo group, resulting in a vaccine efficacy of 93·4% (95% CI: 57·1-99·8). Efficacy against asymptomatic COVID-19 infections was 63·6% (29·0-82·4). In the 1858 elderly participants in the analysis, the split of cases between vaccine and placebo groups was 5 (0·56%) of 893 participants and 16 (1·66%) of 965, respectively, giving an efficacy of 67·8% (8·0-90·0). Efficacy in the 15,115 participants who were younger than 60 years was 79·4% (66·0-88·2).

Further, throat swabs collected from symptomatic or asymptomatic individuals involved in Phase III clinical trial was sequenced and found that individuals were infected with SARS CoV 2 delta variants (B.1.617.2), alpha variant, kappa variant (B.1.617.1) and others and recovered showing the efficacy against other SARS CoV 2 variants. For example, A total of 79 variants were reported from 16,973 samples, 18 in the vaccine and 61 in the placebo group. Among 50 Delta (B.1.617.2) positive-confirmed cases, 13 and 37 participants were in the vaccine and placebo arms, resulting in vaccine efficacy of 65·2% (95% CI: 33·1-83·0). In breakthrough symptomatic Delta variant infections, based on Ct values, the viral load in the vaccine arm was significantly lower than the placebo arm. Efficacy against the Kappa (B.1.617.1) variant was 90·1% (95% CI. 30·4-99·8). No cases of severe variant-related cases of COVID-19 were reported in the vaccines but four severe cases were reported in the placebo recipients infected with Alpha, Kappa, Delta, and unclassified variants respectively.

1. A vaccine formulation for prophylactic vaccine against viral infections, comprising:
(a) a vaccine antigen;
(b) Algel-IMDG as an adjuvant;
(c) preservative; and
(d) a physiologically acceptable buffer.

2. The vaccine formulation as claimed in claim 1, wherein the said vaccine antigen is a whole virion inactivated SARS-CoV-2 or SARS-CoV-2 variants selected form B.1.617.2 (Delta), Brazilian variant (P.1), South African S.501Y.V2 (also known as B.1.351), Japanese Encephalitis (JE), recombinant Hepatitis B surface antigen or Virus like particles (VLPs) such as Human papilloma virus antigen.

3. The vaccine formulation as claimed in claim 2, wherein the said vaccine antigen SARS-CoV-2, SARS-CoV-2 variants or JE is inactivated by beta propiolactone or formaldehyde.

4. The vaccine formulation as claimed in claim 1, wherein the concentration of said vaccine antigen SARS-CoV-2, SARS-CoV-2 variants or JE in the formulation is 1 to 20 μg.

5. The vaccine formulation as claimed in claim 1, wherein Algel-IMDG comprises Al gel as delivery system and Toll-like receptor 7 and Toll-like receptor 8 agonist as a small molecule (IMDG) that can activate immune cells.

6. The vaccine formulation as claimed in claim 5, wherein Al gel is Aluminium hydroxide gel or Aluminium phosphate gel.

7. The vaccine formulation as claimed in claim 5, wherein the Toll-like receptor 7 and Toll-like receptor 8 agonist is meta-amine gallamide N-(3-((4-amino-2-butyl-1H-imidazo[4,5-c]quinolin-1-yl) methyl) benzyl)-3,4,5-trihydroxybenzamide.

8. The vaccine formulation as claimed in claim 5, wherein Algel-IMDG comprises meta-amine gallamide N-(3-((4-amino-2-butyl1H-imidazo[4,5-c]quinolin-1-yl) methyl) benzyl)-3,4,5-trihydroxybenzamide (Imidazoquinoline class molecule), chemisorbed with Aluminium hydroxide gel.

9. The vaccine formulation as claimed in claim 5, wherein said Toll-like receptor 7 and Toll-like receptor 8 agonist with functional groups allow the chemisorption of such compounds to the surface of aluminium hydroxide particles.

10. The vaccine formulation as claimed in claim 8, wherein the Algel-IvDG is prepared by allowing the chemisorption of meta-amine gallamide on to the surface of aluminium hydroxide particles, under continuous stirring up to 72 hrs, allowing the targeted delivery of the Toll-like receptor 7 and Toll-like receptor 8 agonist to draining lymph nodes with negligible systemic exposure, resulting in minimal systemic reactogenicity.

11. The vaccine formulation as claimed in claim 8, wherein the Algel-IMDG is prepared by the method comprising the steps of:
(i) dissolving meta-amine gallamide N-(3-((4-amino-2-butyl-1H-imidazo[4,5-c]quinolin-1-yl) methyl) benzyl)-3,4,5-trihydroxybenzamide in isopropanol;
(ii) keeping the solution of step (i) at 50° C. to dissolve completely;
(iii) filtering the solution of step (ii); and
(iv) adding the solution of N-(3-((4-amino-2-butyl-1H-imidazo[4,5-c]quinolin-1-yl) methyl) benzyl)-3,4,5-tri-hydroxybenzamide obtained from step (iii) to Aluminium hydroxide gel, dropwise under continuous stirring for 72 hours to obtain chemisorbed meta-amine gallamide on to the surface of aluminium hydroxide particles (Algel-IMDG).

12. The vaccine formulation as claimed in claim 1, wherein Algel-IMDG comprises 600-1000 μg of TLR7/8 agonist per ml of Algel-IMDG.

13. The vaccine formulation as claimed in claim 1, wherein Algel-IMDG comprises 250-750 μg of Al³⁺ concentration per dose in 0.5 ml.

14. The vaccine formulation as claimed in claim 1, wherein Algel-IMDG comprises 15-25 μg of TLR7/8 agonist per dose in 0.5 ml.

15. The vaccine formulation as claimed in claim 1, wherein the preservative is Thimerosal or 2-phenoxy ethanol.

16. The vaccine formulation as claimed in claim 15, wherein the concentration of Thimerosal in the formulation is 0.003 to 0.01%.

17. The vaccine formulation as claimed in claim 15, wherein the concentration of 2-phenoxy ethanol in the formulation is 1 to 5 mg/ml.

18. The vaccine formulation as claimed in claim 1, wherein the buffer is phosphate or citrate.

19. The vaccine formulation as claimed in claim 1, wherein the said formulation is stable for 12 months at 2-8° C., 6 months at 25±2° C. and up to 14 days at 37±0.2° C.

20. The vaccine formulation as claimed in claim 1, wherein the formulation provides long-term protective immunity up to 7 months (6 months, post 2^{nd} dose) to the virus by generation of B and T cell memory responses in the vaccinated individuals.

21. The vaccine formulation as claimed in claim 1, wherein the said formulation provides cross neutralization against SARS-CoV-2 variants such as homologous strain (D614G) and heterologous strains such as B.1.128.2, B.1.351, B.1.1.7, B.617, B.617.2.

22. The vaccine formulation as claimed in claim 1, wherein the formulation is used for prophylactic or therapeutic purposes.

23. The vaccine formulation as claimed in claim 6, wherein Algel-IMDG comprises meta-amine gallamide N-(3-((4-amino-2-butyl1H-imidazo[4,5-c]quinolin-1-yl) methyl) benzyl)-3,4,5-trihydroxybenzamide (Imidazoquinoline class molecule), chemisorbed with Aluminium hydroxide gel.

24. The vaccine formulation as claimed in claim 7, wherein Algel-IMDG comprises meta-amine gallamide N-(3-((4-amino-2-butyl1H-imidazo[4,5-c]quinolin-1-yl) methyl) benzyl)-3,4,5-trihydroxybenzamide (Imidazoquinoline class molecule), chemisorbed with Aluminium hydroxide gel.

25. The vaccine formulation as claimed in claim 6, wherein said Toll-like receptor 7 and Toll-like receptor 8 agonist with functional groups allow the chemisorption of such compounds to the surface of aluminium hydroxide particles.

26. The vaccine formulation as claimed in claim 7, wherein said Toll-like receptor 7 and Toll-like receptor 8 agonist with functional groups allow the chemisorption of such compounds to the surface of aluminium hydroxide particles.

27. The vaccine formulation as claimed in claim 8, wherein said Toll-like receptor 7 and Toll-like receptor 8 agonist with functional groups allow the chemisorption of such compounds to the surface of aluminium hydroxide particles.

28. The vaccine formulation as claimed in claim 23, wherein the Algel-IMDG is prepared by allowing the chemisorption of meta-amine gallamide on to the surface of aluminium hydroxide particles, under continuous stirring up to 72 hrs, allowing the targeted delivery of the Toll-like receptor 7 and Toll-like receptor 8 agonist to draining lymph nodes with negligible systemic exposure, resulting in minimal systemic reactogenicity.

29. The vaccine formulation as claimed in claim 24, wherein the Algel-IMDG is prepared by allowing the chemisorption of meta-amine gallamide on to the surface of aluminium hydroxide particles, under continuous stirring up to 72 hrs, allowing the targeted delivery of the Toll-like receptor 7 and Toll-like receptor 8 agonist to draining lymph nodes with negligible systemic exposure, resulting in minimal systemic reactogenicity.

30. The vaccine formulation as claimed in claim 23, wherein the Algel-IMDG is prepared by the method comprising the steps of:
(i) dissolving meta-amine gallamide N-(3-((4-amino-2-butyl-1H-imidazo[4,5-c]quinolin-1-yl) methyl) benzyl)-3,4,5-trihydroxybenzamide in isopropanol;
(ii) keeping the solution of step (i) at 50° C. to dissolve completely;
(iii) filtering the solution of step (ii); and
(iv) adding the solution of N-(3-((4-amino-2-butyl-1H-imidazo[4,5-c]quinolin-1-yl) methyl) benzyl)-3,4,5-tri-hydroxybenzamide obtained from step (iii) to Aluminium hydroxide gel, dropwise under continuous stirring for 72 hours to obtain chemisorbed meta-amine gallamide on to the surface of aluminium hydroxide particles (Algel-IMDG).

31. The vaccine formulation as claimed in claim 24, wherein the Algel-IMDG is prepared by the method comprising the steps of:
(i) dissolving meta-amine gallamide N-(3-((4-amino-2-butyl-1H-imidazo[4,5-c]quinolin-1-yl) methyl) benzyl)-3,4,5-trihydroxybenzamide in isopropanol;
(ii) keeping the solution of step (i) at 50° C. to dissolve completely;
(iii) filtering the solution of step (ii); and
(iv) adding the solution of N-(3-((4-amino-2-butyl-1H-imidazo[4,5-c]quinolin-1-yl) methyl) benzyl)-3,4,5-tri-hydroxybenzamide obtained from step (iii) to Aluminium hydroxide gel, dropwise under continuous stirring for 72 hours to obtain chemisorbed meta-amine gallamide on to the surface of aluminium hydroxide particles (Algel-IMDG).
